(19) United States
(12) Patent Application Publication
(10) Pub. No.: US 2006/0079683 A1
(43) Pub. Date: Apr. 13, 2006

(54) Arylthiobenzylpiperidine derivatives

(76) Inventors: Mohammad R. Marzabadi, Ridgewood, NJ (US); Yu Jiang, Jersey City, NJ (US); Chien-An Chen, Flushing, NY (US); Kai Lu, Lake Hiawatha, NJ (US); Kim Andersen, Ridgewood, NJ (US)

Correspondence Address: LUNDBECK RESEARCH USA, INC.;ATTENTION: STEPHEN G. KALINCHAK, LEGAL, 215 COLLEGE ROAD, PARAMUS, NJ, 07652, US

(21) Appl. No.: 10/961,432

(22) Filed: Oct. 8, 2004

(51) Int. Cl. C07D 413/14; C07D 401/14

(52) U.S. Cl. 544/124; 544/360; 546/194

(57) ABSTRACT

This invention is directed to Arylthiobenzylpiperidine derivatives which are ligands at the MCH1 receptor. The invention provides a pharmaceutical composition comprising a therapeutically effective amount of a compound of the invention and a pharmaceutically acceptable carrier. This invention also provides a pharmaceutical composition made by admixing a therapeutically effective amount of a compound of the invention and a pharmaceutically acceptable carrier. This invention further provides a process for making a pharmaceutical composition comprising combining a therapeutically effective amount of a compound of the invention and a pharmaceutically acceptable carrier. This invention also provides a method of treating a subject suffering from depression and/or anxiety which comprises administering to the subject an amount of a compound of the subject invention. This invention also provides a method of treating a subject suffering from obesity which comprises administering to the subject an amount of a compound of the subject invention.

### FIELD OF THE INVENTION

The present invention relates to compounds that are ligands at the MCH1 receptor, and as such are useful to treat depression, anxiety or obesity.

### BACKGROUND OF THE INVENTION

Throughout this application, various publications are referenced to in full citations. The disclosures of these publications are hereby incorporated by reference into this application to describe more fully the state of the art to which this invention pertains.

Melanin-concentrating hormone (MCH) is a cyclic 19-amino acid peptide produced by neurons in the lateral hypothalamus and zona incerta of the brain. Mammalian MCH is conserved between rat, mouse, and human, exhibiting 100% amino acid homology, and the effects of MCH are mediated through receptors that belong in the rhodopsin superfamily of G protein-coupled receptors. Presently, two receptor subtypes for MCH have been identified in humans, MCH1 and MCH2.

The link between MCH1 and the effects of MCH on feeding was suggested by reports on the phenotype of the MCH1 knockout mice. Independent groups generated knock-out mice with the targeted deletion of the MCH1 receptor. The phenotype of these mice was lean, hyperphagic and hypermetabolic, with increased resistance to diet-induced obesity (D. J. Marsh, et al., Proc. Natl. Acad. Sci 2002, 99, 3240-3245). These observations evidence that MCH1 antagonists are useful to treat obesity.

To further assess the physiological role of the MCH1 receptor, SNAP-7941, a selective MCH1 small molecule antagonist, was evaluated in several animal models (B. Borowsky, et al., Nature Medicine, 2002, 8, 825-830). Pharmacological blockade of the MCH1 receptor with SNAP-7941 produced a profile similar to clinically used anti-depressants and anxiolytics in behavioral models of depression and/ or anxiety: the rat forced-swim, rat social interaction and guinea pig maternal-separation vocalization tests. These observations evidence that MCH1 antagonists are useful to treat depression and anxiety.

Current treatments for depression, anxiety and obesity are on the market. However, numerous patients do not respond to current treatments. Hence, there remains the need for alternative methods of treatment.

### SUMMARY OF THE INVENTION

The objective of the present invention is to provide compounds that are ligands at the MCH1 receptor. The present invention relates to compounds of Fornula I. 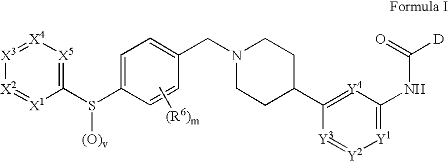
wherein each X¹, X², X³, X⁴ and X⁵ is independently CR¹ or N, provided that if one X is N then the remaining X are each CR¹;
wherein each y¹, y², y³ and y⁴ is independently CR⁷ or N, provided that if one Y is N then the remaining Y are each CR⁷;
wherein D is composed of the following moieties: 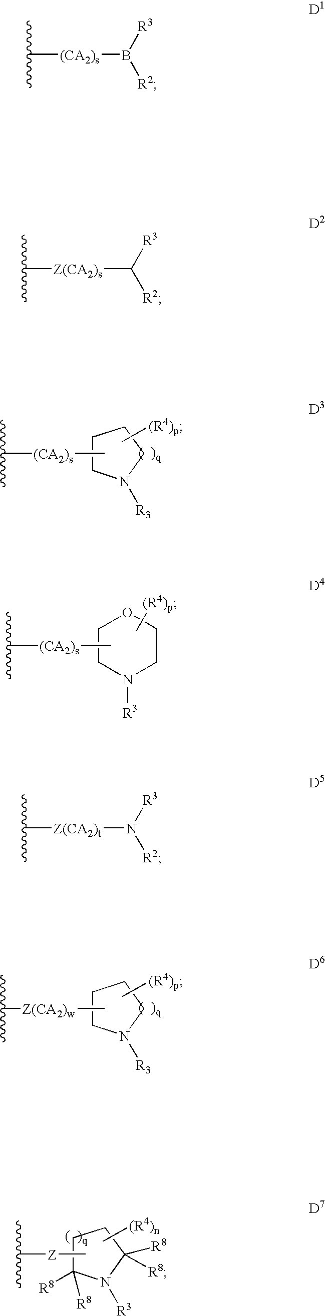 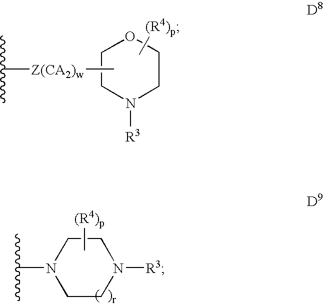
   wherein Z is —N(R⁵) or —O—;
wherein each A is independently H or straight chained or branched C₁-C₄ alkyl;
wherein B is CH or N;
wherein each R¹ is independently H, straight chained or branched C₁-C₇ alkyl, straight chained or branched C₁-C₇ fluoroalkyl, straight chained or branched C₁-C₇ alkoxy, F, Cl, Br or I;
wherein R² is H or straight chained or branched C₁-C₄ alkyl;
wherein R³is H or straight chained or branched C₁-C₄ alkyl;
or wherein if B is N, then the R² moiety, B, the R³ moiety and a bond formed between the R² moiety and the R³ moiety form 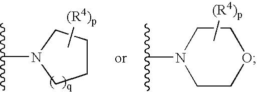
   or wherein if B is CH, then the R² moiety, B, the R³ moiety and a bond formed between the R² moiety and the R³ moiety form cyclopropyl, cyclobutyl, cyclopentyl or cyclohexyl;
wherein R⁴is H, straight chained or branched C₁-C₄ alkyl, straight chained or branched C₁-C₄ fluoroalkyl or F;
wherein R⁵is H or straight chained or branched C₁-C₄ alkyl;
wherein each R⁶ is independently straight chained or branched C₁-C₇ alkyl, straight chained or branched C₁-C₇ fluoroalkyl, straight chained or branched C₁-C₇ alkoxy, F, Cl, Br or I;
wherein each R⁷ is independently H, straight chained or branched C₁-C₇ alkyl, straight chained or branched C₁-C₇ fluoroalkyl, straight chained or branched C₁-C₇ alkoxy, F, Cl, Br or I;
wherein R⁸ is H, straight chained or branched C₁-C₄ alkyl, straight chained or branched C₁-C₄ fluoroalkyl or F;
wherein m is an integer from 0 to 4 inclusive;
wherein n is an integer from 0 to 2 inclusive;
wherein p is an integer from 0 to 4 inclusive;
wherein q is an integer from 0 to 3 inclusive;
wherein r is 1 or 2;
wherein s is an integer from 0 to 4 inclusive;
wherein t is an integer from 2 to 4 inclusive;
wherein v is an integer from 0 to 2 inclusive; and
wherein w is an integer from 1 to 5 inclusive;
or a pharmaceutically acceptable salt thereof.

In separate embodiments of the invention, the compound is selected from one of the specific compounds disclosed in the Experimental Section.

Furthermore, the present invention provides a pharmaceutical composition comprising a therapeutically effective amount of a compound of Formula I and a pharmaceutically acceptable carrier. The present invention also provides a process for making a pharmaceutical composition comprising admixing a compound of Formula I and a pharmaceutically acceptable carrier.

Moreover, the present invention provides a method of treating a subject suffering from depression comprising administering to the subject a therapeutically effective amount of a compound of Formula I. The present invention further provides a method of treating a subject suffering from anxiety comprising administering to the subject a therapeutically effective amount of a compound of Formula I.

### DETAILED DESCRIPTION OF THE INVENTION

Definitions

In the present invention, the term “straight chained or branched C₁-C₇ alkyl” refers to a saturated hydrocarbon having from one to seven carbon atoms inclusive. Examples of such substituents include, but are not limited to, methyl, ethyl, 1-propyl, 2-propyl, 1-butyl, 2-butyl, 2-methyl-2-propyl and 2-methyl-1-propyl. Similarly, the term “straight chained or branched C₁-C4 alkyl” refers to a saturated hydrocarbon having from one to four carbon atoms inclusive.

The term “straight chained or branched C₁-C₇ fluoroalkyl” refers to a saturated hydrocarbon having from one to seven carbon atoms inclusive substituted with one or more fluorine atoms. Examples of such substituents include, but are not limited to, trifluoromethyl, pentafluoroethyl, 1-fluoroethyl and 1,2-difluoroethyl. Similarly, the term “straight chained or branched C₁-C₄ fluoroalkyl” refers to a saturated hydrocarbon having from one to four carbon atoms inclusive substituted with one or more fluorine atoms per carbon atom.

The term “straight chained or branched C₁-C₇ alkoxy” refers to a saturated alkoxy group having from one to seven carbon atoms inclusive with the open valency on the oxygen. Examples of such substituents include, but are not limited to, methoxy, ethoxy, n-butoxy and t-butoxy.

The specific compounds disclosed in the present invention are identified by their IUPAC names. The names of the compounds were generated using the program Chemistry 4-D Draw Nomenclator™ Database (Version 7.01c, Cheminnovation Software, Inc.). According to Cheminnovation Software Inc., Nomenclator™ automatically assigns systematic names to organic structures according to IUPAC nomenclature rules. Accordingly, this application discloses the Arylthiobenzylpiperidine derivatives encompassed by Formula I in accordance with IUPAC nomenclature rules.

For illustrative purposes, and without limiting the invention, the compound of example 1k has the following structure:

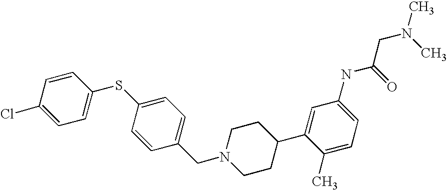

This compound is constructed from Formula I wherein each X¹, X², X³, X⁴ and X⁵ is CR¹; wherein each R¹ is independently H or Cl; wherein v is 0; wherein m is 0; wherein each Y¹, y², y³ and y⁴ is CR⁷; wherein each R⁷ is independently H or methyl; wherein D is D¹; wherein s is 1; wherein each A is H; wherein B is N; wherein R² is methyl and wherein R³ is methyl.

Additionally, the invention further provides certain embodiments of the present invention that are described below.

In one embodiment of the invention of Formula I, m is 0 or 1, and R⁶ is methyl, F or Cl.

In another embodiment, each X¹, X², X³, X⁴ and X⁵ is CR¹, and each y¹, y², y³ and y⁴ is CR⁷.

In another embodiment, each R¹ is independently H, methyl, F or Cl, and each R⁷ is independently H, F or methyl.

In another embodiment, D is

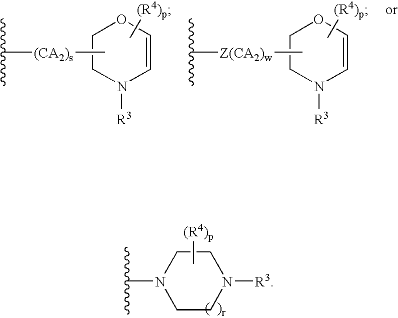

In another embodiment, p is 0 and R₃ is H or methyl.

In another embodiment, D is

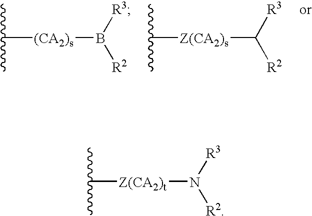

In another embodiment, D is

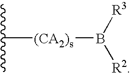

In another embodiment, if B is N, then the R² moiety, B, the R³ moiety and the bond formed between the R² moiety and the R³ moiety form: 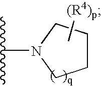
or wherein B is CH then the R² moiety, B, the R³ moiety and the bond formed between the R² moiety and the R³ moiety form cyclopropyl, cyclobutyl, cyclopentyl or cyclohexyl;

In another embodiment, B is N, and R² and R³ are independently H, methyl or ethyl.

In another embodiment, s is 1 or 2.

In another embodiment, D is

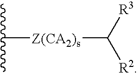

In another embodiment, R² and R³ are independently H, methyl or ethyl.

In another embodiment, D is

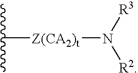

In another embodiment, the R² moiety, N, the R³ moiety and the bond formed between the R² moiety and the R³ moiety form:

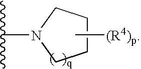

In another embodiment, R² and R³ are independently H, methyl or ethyl.

In another embodiment, D is 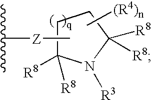
n is 0and R³ is H or methyl.

In another embodiment, D is

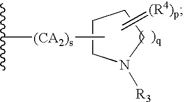

In another embodiment, s is 0 or 1.

In another embodiment, D is 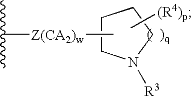
p is 0 and R³ is H or methyl.
Pharmaceutically Acceptable Salts

The present invention also comprises salts of the present compounds, typically, pharmaceutically acceptable salts. Such salts include pharmaceutically acceptable acid addition salts. Acid addition salts include salts of inorganic acids as well as organic acids.

Representative examples of suitable inorganic acids include hydrochloric, hydrobromic, hydroiodic, phosphoric, sulfuric, sulfamic, nitric acids and the like. Representative examples of suitable organic acids include formic, acetic, trichloroacetic, trifluoroacetic, propionic, benzoic, cinnamic, citric, fumaric, glycolic, itaconic, lactic, methanesulfonic, maleic, malic, malonic, mandelic, oxalic, picric, pyruvic, salicylic, succinic, methane sulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, theophylline acetic acids, as well as the 8-halotheophyllines, for example 8-bromotheophylline and the like. Further examples of pharmaceutically acceptable inorganic or organic acid addition salts include the pharmaceutically acceptable salts listed in S. M. Berge, et al., J. Pharm. Sci. 1977, 66, 2, the contents of which are hereby incorporated by reference.

Furthermore, the compounds of this invention may exist in unsolvated as well as in solvated forms with pharmaceutically acceptable solvents such as water, ethanol and the like. In general, the solvated forms are considered equivalent to the unsolvated forms for the purposes of this invention.

Racemic forms may be resolved into the optical antipodes by known methods, for example, by separation of diastereomeric salts thereof with an optically active acid, and liberating the optically active amine compound by treatment with a base. Separation of such diastereomeric salts can be achieved, e.g. by fractional crystallization. The optically active acids suitable for this purpose may include, but are not limited to d- or l-tartaric, madelic or camphorsulfonic acids. Another method for resolving racemates into the optical antipodes is based upon chromatography on an optically active matrix. The compounds of the present invention may also be resolved by the formation and chromatographic separation of diastereomeric derivatives from chiral derivatizing reagents, such as, e.g., chiral alkylating or acylating reagents, followed by cleavage of the chiral auxiliary. Any of the above methods may be applied either to resolve the optical antipodes of the compounds of the invention per se or to resolve the optical antipodes of synthetic intermediates, which can then be converted by methods described herein into the optically resolved final products which are the compound of the invention.

Additional methods for the resolution of optical isomers, known to those skilled in the art, may be used. Such methods include those discussed by J. Jaques, A. Collet and S. Wilen in Enantiomers, Racemates, and Resolutions, John Wiley and Sons, New York 1981. Optically active compounds were also be prepared from optically active starting materials.

The invention also encompasses prodrugs of the present compounds, which on administration undergo chemical conversion by metabolic processes before becoming pharmacologically active substances. In general, such prodrugs will be functional derivatives of the compounds of Formula I which are readily convertible in vivo into the required compound of Formula I. Conventional procedures for the selection and preparation of suitable prodrug derivatives are described in Design of Prodrugs, ed. H. Bundgaard, Elsevier, 1985.

Pharmaceutical Compositions

The present invention further provides a pharmaceutical composition comprising a therapeutically effective amount of a compound of Formula I and a pharmaceutically acceptable carrier. The present invention also provides a pharmaceutical composition comprising a therapeutically effective amount of one of the specific compounds disclosed in the Experimental Section and a pharmaceutically acceptable carrier.

The compounds of the invention may be administered alone or in combination with pharmaceutically acceptable carriers or excipients, in either single or multiple doses. The pharmaceutical compositions according to the invention may be formulated with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

The pharmaceutical compositions may be specifically formulated for administration by any suitable route such as oral, rectal, nasal, pulmonary, topical (including buccal and sublingual), transdermal, intracisternal, intraperitoneal, vaginal and parenteral (including subcutaneous, intramuscular, intrathecal, intravenous and intradermal) routes. It will be appreciated that the route will depend on the general condition and age of the subject to be treated, the nature of the condition to be treated and the active ingredient.

Pharmaceutical compositions for oral administration include solid dosage forms such as capsules, tablets, dragees, pills, lozenges, powders and granules. Where appropriate, the compositions may be prepared with coatings such as enteric coatings or they may be formulated so as to provide controlled release of the active ingredient such as sustained or prolonged release according to methods well known in the art. Liquid dosage forms for oral administration include solutions, emulsions, suspensions, syrups and elixirs.

Pharmaceutical compositions for parenteral administration include sterile aqueous and nonaqueous injectable solutions, dispersions, suspensions or emulsions as well as sterile powders to be reconstituted in sterile injectable solutions or dispersions prior to use.

Other suitable administration forms include, but are not limited to, suppositories, sprays, ointments, creams, gels, inhalants, dermal patches and implants.

Typical oral dosages range from about 0.001 to about 100 mg/kg body weight per day. Typical oral dosages also range from about 0.01 to about 50 mg/kg body weight per day. Typical oral dosages further range from about 0.05 to about 10 mg/kg body weight per day. Oral dosages are usually administered in one or more dosages, typically, one to three dosages per day. The exact dosage will depend upon the frequency and mode of administration, the sex, age, weight and general condition of the subject treated, the nature and severity of the condition treated and any concomitant diseases to be treated and other factors evident to those skilled in the art.

The formulations may also be presented in a unit dosage form by methods known to those skilled in the art. For illustrative purposes, a typical unit dosage form for oral administration may contain from about 0.01 to about 1000 mg, from about 0.05 to about 500 mg, or from about 0.5 mg to about 200 mg.

For parenteral routes such as intravenous, intrathecal, intramuscular and similar administration, typical doses are in the order of half the dose employed for oral administration.

The present invention also provides a process for making a pharmaceutical composition comprising admixing a therapeutically effective amount of a compound of Formula I and a pharmaceutically acceptable carrier. In an embodiment of the present invention the compound utilized in the aforementioned process is one of the specific compounds disclosed in the Experimental Section.

The compounds of this invention are generally utilized as the free substance or as a pharmaceutically acceptable salt thereof. One example is an acid addition salt of a compound having the utility of a free base. When a compound of Formula I contains a free base such salts are prepared in a conventional manner by treating a solution or suspension of a free base of Formula I with a molar equivalent of a pharmaceutically acceptable acid. Representative examples of suitable organic and inorganic acids are described above.

For parenteral administration, solutions of the compounds of Formula I in sterile aqueous solution, aqueous propylene glycol, aqueous vitamin E or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The compounds of Formula I may be readily incorporated into known sterile aqueous media using standard techniques known to those skilled in the art.

Suitable pharmaceutical carriers include inert solid diluents or fillers, sterile aqueous solutions and various organic solvents. Examples of solid carriers include lactose, terra alba, sucrose, cyclodextrin, talc, gelatin, agar, pectin, acacia, magnesium stearate, stearic acid and lower alkyl ethers of cellulose. Examples of liquid carriers include, but are not limited to, syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the compounds of Formula I and a pharmaceutically acceptable carrier are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and optionally a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatin capsule in powder or pellet form or it may be in the form of a troche or lozenge. The amount of solid carrier will vary widely but will range from about 25 mg to about 1 g per dosage unit.

If a liquid carrier is used, the preparation may be in the form of a syrup, emulsion, soft gelatin capsule or sterile injectable liquid such as an aqueous or non-aqueous liquid suspension or solution.

Treatment of Disorders

As mentioned above, the compounds of Formula I are ligands at the MCH1 receptor. The present invention provides a method of treating a subject suffering from depression and/or anxiety which comprises administering to the subject a therapeutically effective amount of a compound of this invention. This invention further provides a method of treating a subject suffering from major depression and/or anxiety which comprises administering to the subject a therapeutically effective amount of a compound of this invention. This invention also provides a method of treating a subject suffering from obesity which comprises administering to the subject a therapeutically effective amount of a compound of this invention. In an embodiment of this invention, the subject is a human being.

The invention will be better understood from the Experimental Details which follow. However, one skilled in the art will readily appreciate that the specific methods and results discussed therein are merely illustrative of the invention as described more fully in the claims which follow thereafter. Furthermore, the variables depicted in Schemes 1-12 are consistent with the variables recited in the Summary of the Invention. For clarity purposes, the variables X¹, X², X³, X⁴ and X⁵ are designated as variable X in the experimental schemes. Moreover, the variables y¹, y², y³ and y⁴ are designated as variable Y in the experimental schemes.

In the Experimental Section, standard acronyms are used. Examples of such acronyms include AIBN (2,2′-Azobisisobutyronitrile); DMF (N,N-Dimethylformamide); DMSO (Dimethylsulfoxide); NBS (N-Bromosuccinimide); HATU (O-(7-Azabenzotriazol-1-yl)-N,N,N′,N′-tetramethyluronium hexafluorophosphate); mCPBA (3-chloroperoxybenzoic acid); CbzCl (Benzyl chloroformate); and BOC (tert-butoxycarbonyl). Furthermore in certain instances, the methods of preparing the compounds of the invention are described generally by referring to representative reagents such as bases or solvents. The particular reagent identified is representative but is not inclusive or does not limit the invention in any way. For example, representative bases include but are not limited to K₂CO₃, Et₃N or DIPEA (Diisopropylethylamine).

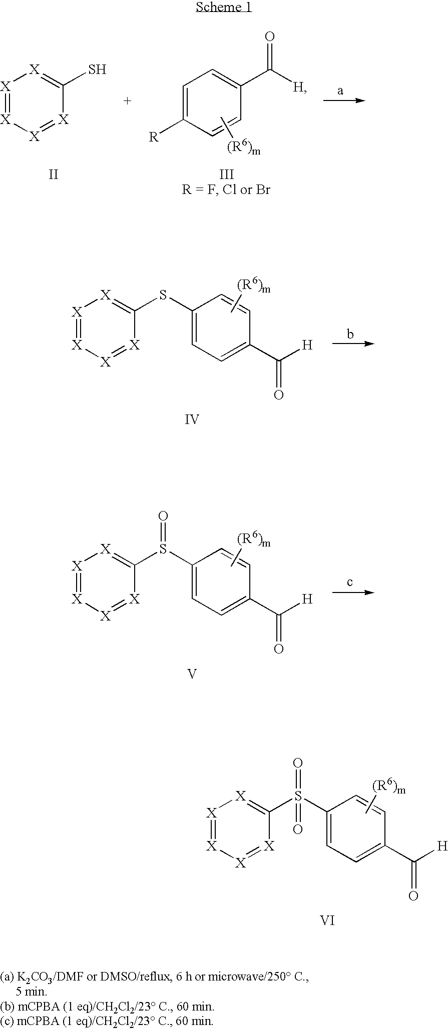

The aldehydes of Formula IV, V and VI, used as starting materials in Scheme 8, are either available from commercial sources or prepared as shown in Scheme 1. The aldehydes of Formula IV are prepared via aromatic nucleophilic reaction of thiophenols II and activated 4-halo-benzaldehydes III in the presence of base under reflux or microwave conditions. Alternatively, the aldehydes of Formula IV may be prepared via Ullmann type reactions (Kondo, T. et al, Chem. Rev. 2000, 100, 3205-3220 and the references cited therein). The corresponding sulfoxides V and sulfones VI are prepared via sequential oxidations of IV by mCPBA as shown in Scheme 1. Alternatively, the aldehydes of Formula VI may be synthesized via a sequence of protection, oxidation and deprotection of the aldehyde IV by using standard conditions.

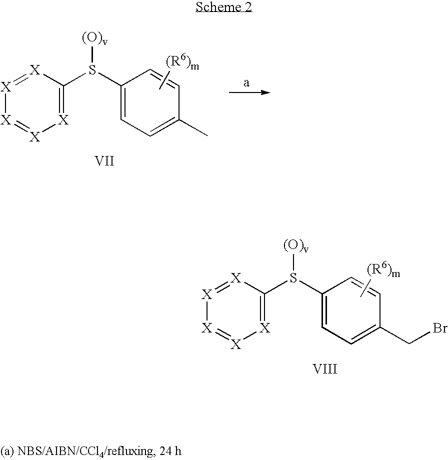

The benzylbromides of Formula VIII are used as starting material in Scheme 9, are either available from commercial sources or prepared via bromination reactions from the corresponding 4-methyl-benzenes VII in the presence of NBS under reflux as shown in Scheme 2.

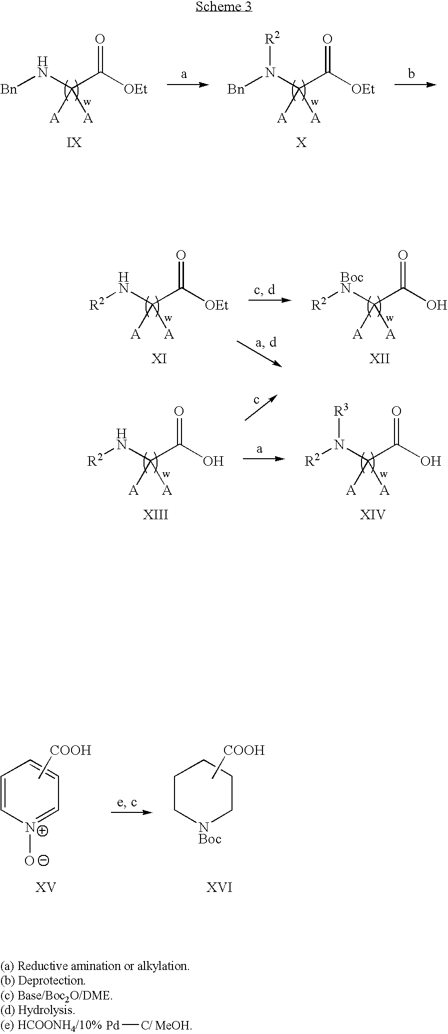

The N-protected primary or secondary amino acids XII, tertiary amino acids XIV and N-protected piperidine carboxylic acids XVI used as starting materials in Scheme 10, and are either commercially available or prepared according to literature procedures or as outlined in Scheme 3. For example, the N-protected amino acids XII and tertiary amino acids XIV are prepared from the corresponding ester XI, X, XI or carboxylic acid XIII. The N-protected piperidine carboxylic acids XVI may be prepared by reduction of the corresponding substituted pyridine or pyridine N-oxide XV, followed by Boc protection as shown in Scheme 3. (For representative reviews for the preparation of optically active α-amino acids, see: R. M. Williams, In Synthesis of Optically Active α-Amino Acids, J. E. Baldwin, Ed.; Organic Chemistry Series, Pergamon Press: Oxford, 1989; R. M. Williams, Chem. Rev. 1992, 92, 889; R. 0. Duthaler, Tetrahedron 1994, 50, 1539; C. Cativiela, Tetrahedron: Asymmetry 1998, 9, 3517; C. Cativiela, Tetrahedron: Asymmetry 2000, 11, 645; M. J. O'Donnell, Aldrichimica Acta 2001, 3, 3-15; Enzyme Catalysis in Organic Synthesis; K. Drauz, H. Waldmann, Eds.; Wiley-VCH: Weinheim, 1995; Stereoselective Biocatalysis; R. N. Patel, Ed.; Marcel Dekker, New York, 2000; and K. Maruoka, Chem. Rev. 2003, 103, 3013-3028. For representative reviews on the preparation of optically active β-amino acids, see: Enantioselective Synthesis of β-Amino Acids; E. Juaristi, Wiley-VCH, New York, 1997; M. P. Sibi, Tetrahedron 2002, 58, 7991-8035; D. C. Cole, Tetrahedron 1994, 50, 9517-9582; E. Juaristi, Aldrichim. Acta 1994, 27, 3; G. Cardillo, Chem. Soc. Rev. 1996, 25,117-128; Y. Yamamoto, N. Asgo and W. Tsukada, Advances in Asymmetric Synthesis (Ed.: A. Hassner), JAI Press, Stamford, 1998, p. 1. For the preparation of azepane carboxylic acids, see G. I. Georg et al., Bioorg. Med. Chem. Lett. 1991, 1, 125-128. For the preparation of piperidine carboxylic acids, see B. Zacharie et al., J. Org. Chem. 2001, 66, 5264-5265. For the preparation of pyrrolidine carboxylic acids, see R. Ling et al., Tetrahedron 2001, 57, 6579-6588; B.C.J. van Esseveldt et al., SynLett 2003, 15, 2354-2358. For the preparation of azetidine carboxylic acids, see S. Hanessian et al., Bioorg. Med. Chem. Lett. 1999, 9, 1437-1442; R. A. Miller et al., Synth. Commun. 2003, 33, 3347-3353 and references therein).

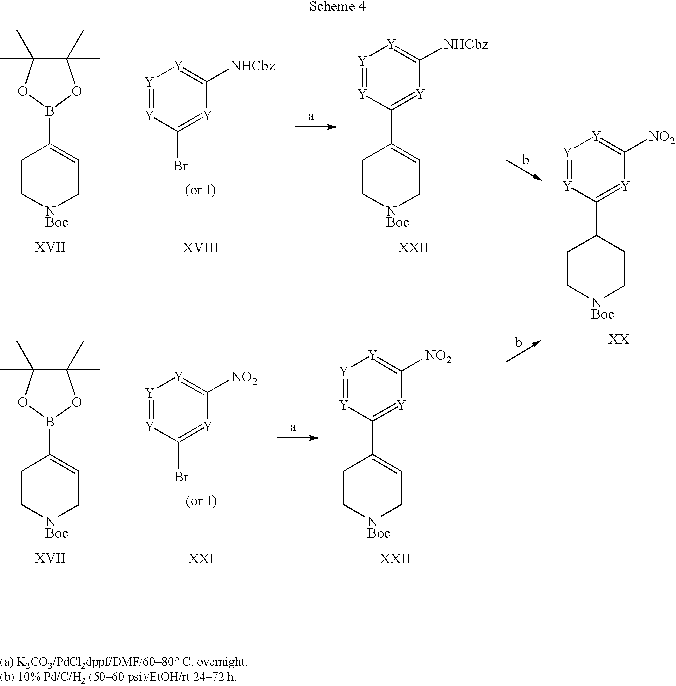

Intermediate tert-butyl 4-(3-aminoaryl)piperidinecarboxylates of Formula XX are prepared as outlined in Scheme 4 from tert-butyl 4-(4,4,5,5-tetramethyl-1,3,2-dioxaborolan-2-yl)-1,2,5,6-tetrahydropyridine carboxylate XVII and N-Cbz protected bromo or iodo anilines or amino pyridines XVIII via Suzuki coupling followed by simultaneous reduction of the double bond in the tetrahydropyridine ring and removal of the Cbz protecting group by catalytic hydrogenation. Altematively, tert-butyl 4-(3-aminoaryl)piperidinecarboxylate XX may be prepared from tert-butyl 4-(4,4,5,5-tetramethyl-1,3,2-dioxaborolan-2-yl)-1,2,5,6-tetrahydropyridine carboxylate XVII and bromo or iodo nitrobenzenes or nitropyridines XXI via Suzuki coupling followed by simultaneous reduction of the double bond and the nitro group by means of catalytic hydrogenation. (Suzuki coupling and hydrogenation reactions are described in the following references: A. Suzuki et al, Chem. Rev. 1995, 95, 2457; A. Suzuki, J. Organomet Chem. 1999, 576, 147-168 and the references cited therein; and P. N. Rylander, Hydrogenation Methods (Best Synthetic Methods Series), Academic Press, 1990). tert-Butyl 4-(4,4,5,5-tetramethyl-1,3,2-dioxaborolan-2-y)-1,2,5, 6-tetrahydropyridine carboxylate XVII used as starting material in Scheme 4 was prepared according to the procedures described by P. R. Eastwood, Tetrahedron Lett. 2000, 41, 3705-3708 and references cited therein.

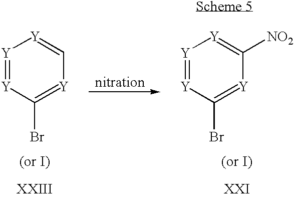

The 3-bromo or 3-iodo nitrobenzenes and nitropyridines XXI which are used as starting material for the synthesis outlined in Scheme 4 are available from commercial sources or may alternatively be prepared from the corresponding bromo or iodo benzenes and pyridines XXIII by nitration methods. General information regarding aromatic nitration is described in the following references: J. G. Hoggett, R. B. Moodie, J. R. Penton and K. Schofield, Nitration and Aromatic Reactivity, Cambridge University Press, London, 1971; K. Schofield, Aromatic Nitration, Cambridge University Press, London, 1980; and G. A. Olah, R. Malhotra and S. C. Narang, Nitration: Methods and Mechanism, (Ed.: H. Feuer), VCH Publishers, New York, 1989.

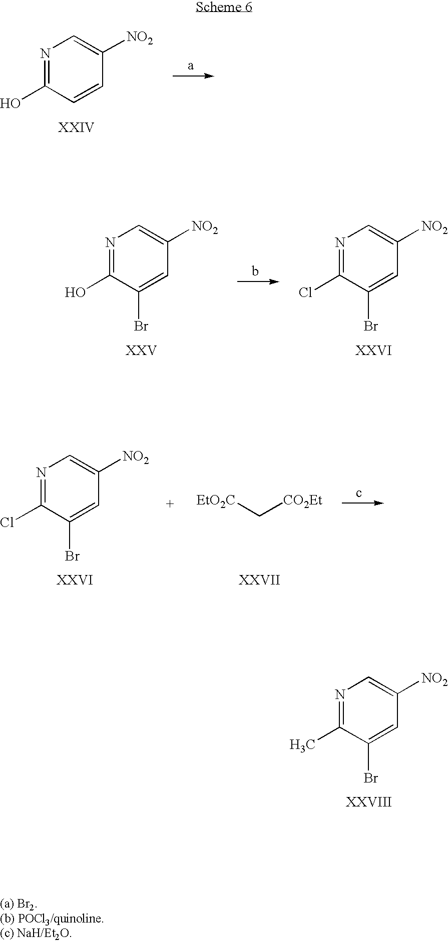

Alternatively, the substituted bromo or iodo nitrobenzenes or nitropyridines XXI may be prepared from commercially available materials via a series of functional group transformation methods known to those skilled in the art. For example, 3-bromo-2-methyl-5-nitropyridine XXVIII may be prepared and functionalized from 5-nitropyridin-2-ol XXIV as shown in Scheme 6.

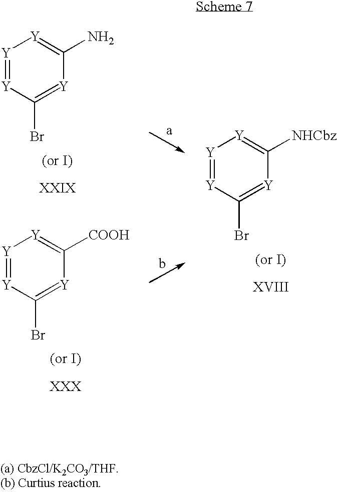

The N-Cbz bromo or iodo anilines and amino pyridine XVIII which are used as starting material in the syntheses outlined in Scheme 4 may be prepared by a variety of conditions from commercially available materials. For example, the amino group of commercially available bromo or iodo anilines and amino pyridines XXIX may be protected directly by benzyl chloroformate in the presence of base. Alternatively, N-Cbz protected bromo or iodo anilines and amino pyridines XVIII may be prepared from the corresponding benzoic acids, isonicotinic acids, nicotinic acids or picolinic acids XXX using diphenylphosphoryl azide via a Curtius type rearrangement, followed by trapping the isocyanates with benzyl alcohol as described by S. Yamada et al., Tetrahedron 1974, 30, 2151-2157.

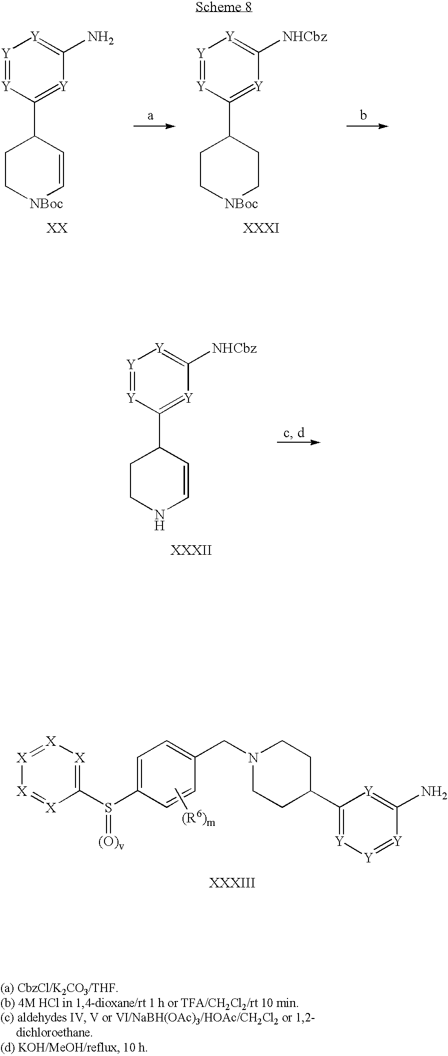

The intermediates of Formula XXXIII are prepared as shown in Scheme 8. tert-Butyl 4-(3-aminoaryl)piperidinecarboxylate XX is acylated with CbzCl in the presence of base to afford tert-butyl 4-{3-[(phenylmethoxy)carbonylamino]aryl}piperidinecarboxylate XXXI. The Boc protecting group is removed under acidic conditions to give N-(3-(4-piperidyl)aryl)(phenylmethoxy)carboxamide XXXII. Reductive amination of piperidine XXXII with a variety of benzaldehydes of Formulas IV, V and VI using sodium triacetoxyborohydride followed by removal of the Cbz group under basic conditions affords arylamines XXXIII.

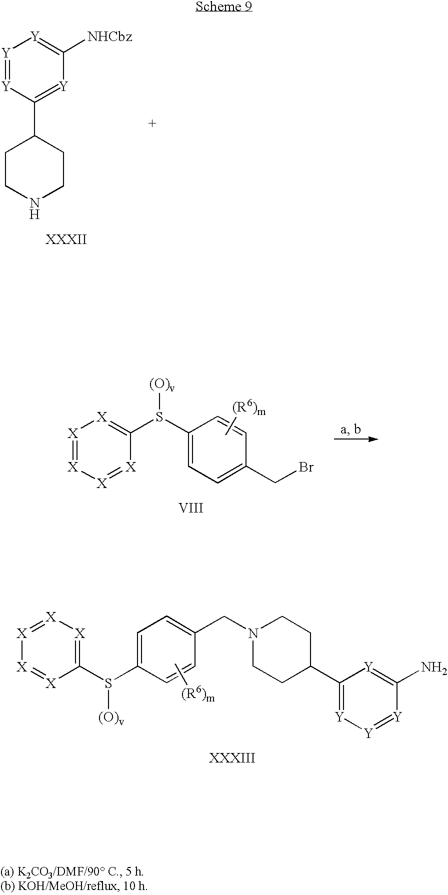

Alternatively the arylamines XXXIII may be prepared via alkylations of piperidines XXXII with benzyl bromides Vil under basic conditions followed by hydrolysis to remove the Cbz group.

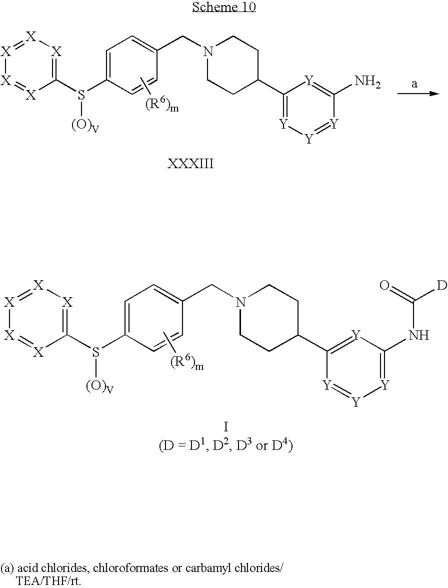

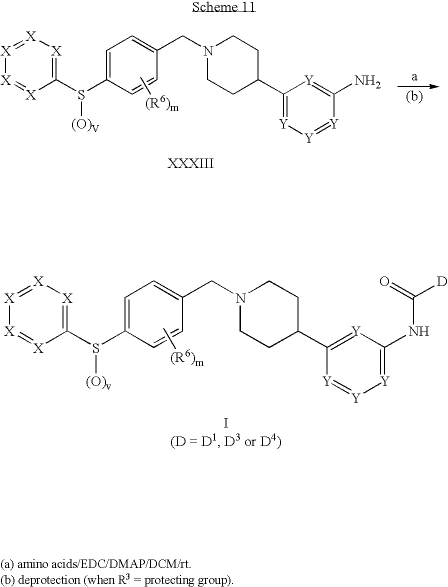

Compounds of the invention of Formula I (D=D¹, D², D³ or D⁴) are prepared as outlined in Scheme 10. 3-{1-[(4-arylthiophenyl)methyl]-4-piperidyl}arylamines XXXIII are acylated with acid chlorides, chloroformates or carbamyl chlorides under standard coupling conditions to give compounds of Formula I (D=D¹, D³ or D⁴) Compounds of the invention of Formula I (D=D¹, D², D³ or D⁴) are prepared as outlined in Scheme 11. 3-{1-[(4-arylthiophenyl)methyl]-4-piperidyl}arylamines XXXIII are acylated with N-protected amino acids (for primary and secondary amino acids) or amino acids (for tertiary amino acids) to provide the amide derivatives. For primary or secondary amino acids (R² or R³=H) the protecting groups are removed using standard conditions. Compounds of the invention of Formula I (D=D², D⁵, D⁶, D⁷, D⁸ or D⁹) are prepared as outlined in Scheme 12. The ureas and carbamates of Formula I (D=D², D⁵, D⁶, D⁷, D⁸ or D⁹) are prepared by the reaction of an amine or an alcohol (N-protected if necessary) with 3-{1-[(4-arylthiophenyl)methyl]-4-piperidyl}arylisocyanate XXXIV and N-[3-(1-{[4-arylthiophenyl]methyl}(4-piperidyl))aryl](4-nitrophenoxy)carboxamide XXXV, followed by removal of the protecting group (for R³ or R²=H) to give amines of Formula I (D=D², D⁵, D⁶, D⁷, D⁸ or D⁹).

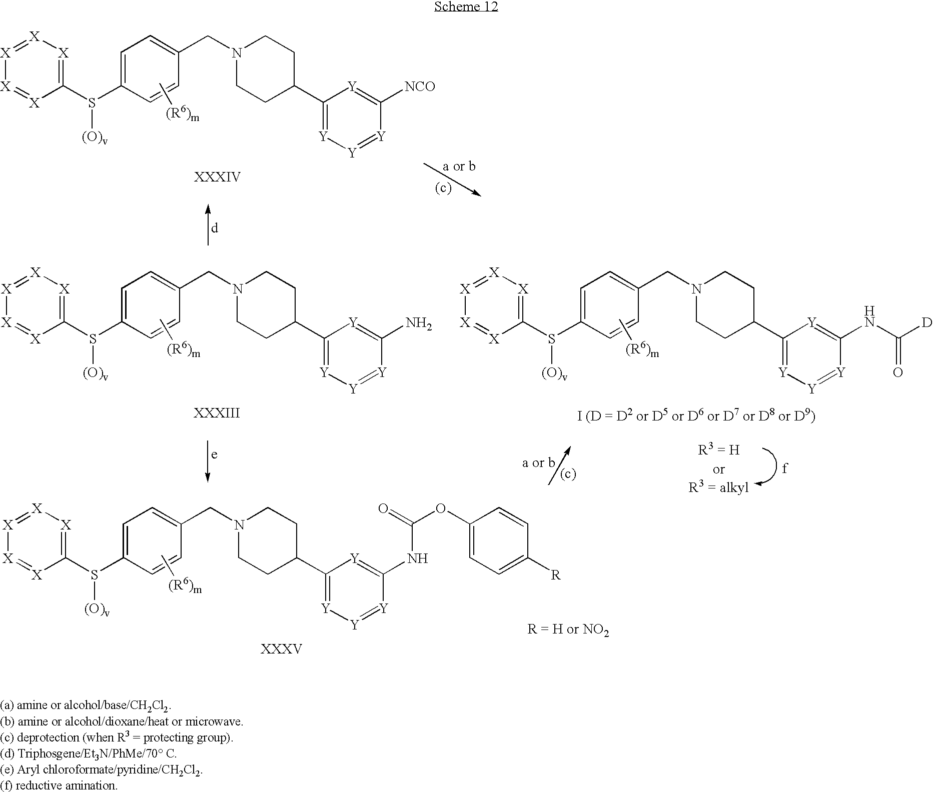

The 3-{1-[(4-arylthiophenyl)methyl]-4-piperidyl}arylisocyanates XXXIV are prepared from XXXIII by using triphosgene under standard conditions. The activated phenyl carbamates of Formula XXXV are prepared from XXXIII under standard conditions.

The primary and secondary amines with Formula I (R³=H) may be further converted to tertiary amines (R³=alkyl) via a reductive amination procedure. Any modification of the sequence in the schemes including the use of other protective groups or different conditions for amide, urea, carbamate formation would be apparent to those skilled in the art. The general information for protecting/deprotecting the amino group can be found in the textbook (T. Green and P. G. M. Wuts, Protective Groups in Organic Synthesis, John Wiley & Sons, Inc., New York, N.Y., 1999).

General Methods: All reactions were performed under a nitrogen atmosphere and the reagents, neat or in appropriate solvents, were transferred to the reaction vessel via syringe and cannula techniques. Anhydrous solvents were purchased from Aldrich Chemical Company and used as received. The NMR spectra were recorded at Bruker Avance (400 MHz) or GE QEPlus300 in CDCl₃, MeOH-d₄ or DMSO-d₆ as solvent with tetramethylsilane as the internal standard unless otherwise noted. Chemical shifts (δ) are expressed in ppm, coupling constants (J) are expressed in Hz, and splitting patterns are described as follows: s=singlet; d=doublet; t=triplet; q=quartet; quintet; sextet; septet; br=broad; m=multiplet; dd=doublet of doublets; dt=doublet of triplets; td=triplet of doublets; dm=doublet of multiplets; ddd=doublet of doublet of doublets. Elemental analyses were performed by Robertson Microlit Laboratories, Inc. Unless otherwise noted, mass spectra were obtained using electrospray ionization (ESMS, Micromass Platform II or Quattro Micro) and (M+H)⁺ is reported. Thin-layer chromatography (TLC) was carried out on glass plates pre-coated with silica gel 60 F₂₅₄ (0.25 mm, EM Separations Tech.). Preparative TLC was carried out on glass sheets pre-coated with silica gel GF (2 mm, Analtech). Flash column chromatography was performed on Merck silica gel 60 (230-400 mesh). Melting points (mp) were determined in open capillary tubes on a Mel-Temp apparatus and are uncorrected. Reverse Phase High Pressure Liquid Chromatography purification was performed using a Genesis HPLC Column (Ref. 16R10985, 100 mm×22.5 mm) containing C18-7 μm, 120 Å silica. Microwave experiments were carried out using a Biotage Emyrs Optimizer™ or Smithcreator.

Preparation of Intermediates

Benzaldehyde Synthesis:

4-(4-chlorophenylthio)benzaldehyde:

A mixture of 4-fluorobenzaldehyde (12.6 mmol, 1.57 g), 4-chlorobenzenethiol (12.6 mmol, 1.81 g), K₂CO₃ (15.1 mmol, 2.09 g) and DMF (5.00 mL) was heated at 90° C. for 10 h. After cooling to room temperature, the reaction mixture was poured into a separatory funnel with 100 mL of water. The phases separated and the aqueous layer was extracted with CH₂Cl₂ (3×50 mL) and the combined organic layers were washed with water (2×100 mL), brine (50 mL) and dried over MgSO₄. Removal of the solvents in vacuo gave a light yellow liquid. Purification by flash chromatography (5% EtOAc in Hexane) provided 4-(4-chlorophenylthio)benzaldehyde (2.00 g, 76.0% yield) as a light yellow liquid. ¹H NMR (CDCl₃) 6 9.93 (s, 1H), 7.72 (d, J=8.3Hz, 2H), 7.46-7.39 (m, 4H), 7.25 (d, J=8.3Hz, 2H).

4-[(4-chlorophenyl)sulfinyl]benzaldehyde:

A mixture of 4-(4-chlorophenylthio)benzaldehyde (3.63 mmol, 900 mg), 3-chloroperoxybenzoic acid (max 77%, 3.63 mmol, 813 mg) and CH₂Cl₂ (5.00 mL), was stirred at room temperature for 60 min and poured into a separatory funnel with 10 mL of 5% KOH solution. The phases were separated and the aqueous layer was extracted with CH₂Cl₂ (3×10 mL) and the combined organic extracts were washed with water (10 mL), brine (50 mL) and dried over MgSO₄. Removal of solvents in vacuo gave a light yellow liquid. Purification by flash chromatography (5% EtOAc in Hexane) provided 4-[(4-chlorophenyl)sulfinyl]benzaldehyde (500 mg, 52.2% yield) as a light yellow solid. ¹H NMR (CDCl₃) 6 10.04 (s, 1H), 7.98 (d, J=8.2Hz, 2H), 7.82 (d, J=8.2Hz, 2H), 7.62 (d, J=8.5Hz, 2H), 7.46 (d, J=8.5Hz, 2H).

4-[(4-chlorophenyl)sulfonyl]benzaldehyde:

A mixture of 4-(4-chlorophenylthio)benzaldehyde (3.63 mmol, 900 mg), 3-chloroperoxybenzoic acid (max 77%, 10.9 mmol, 2.44 g) in CH₂Cl₂ (5.00 mL) was stirred at room temperature for 60 min and poured into a separatory funnel with 20 mL of 5% KOH solution. The phases separated and the aqueous layer was extracted with CH₂Cl₂ (3×10 mL). The combined organic layers were washed with water (10 mL), brine (50 mL) and dried over MgSO₄. Removal of solvents in vacuo gave a light yellow liquid. Purification by flash chromatography (5% EtOAc in Hexane) provided 4-[(4-chlorophenyl)sulfonyl]benzaldehyde (700 mg, 68.9% yield) as a light yellow solid. ¹H NMR (CDCl₃) 6 10.09 (s, 1H) 8.11-8.00 (m, 4H), 7.91 (d, J=8.7Hz, 2H), 7.51 (d, J=8.7Hz, 2H).

Benzyl Bromide Synthesis:

4-(bromomethyl)-1-[(4-methylphenyl)sulfinyl]benzene:

A mixture of 4-methyl-1-[(4-methylphenyl)sulfinyl]benzene (1.15 g, 5.00 mmol), N-bromosuccinimide (1.08 g, 6.00 mmol), 2,2′-azobis-(2-methylpropionitrile) (100 mg, 0.600 mmol), and CCl₄ (25.0 mL) as solvent was stirred for 5 min at room temperature and then heated to reflux for 12 h. The reaction mixture was cooled to room temperature and filtered and the solvent was removed in vacuo, affording a crude product which was used in the next step without any further purification (1.21 g, 78.1%). ¹H NMR (CDCl₃) 6 7.71-7.57 (m, 2H), 7.57-7.40 (m, 4H), 7.36-7.16 (m, 2H), 4.46 (s, 2H), 2.37 (s, 3H).

tert-butyl 4-(3-aminoaryl)piperidinecarboxylate synthesis:

tert-butyl 4-(4,4,5,5-tetramethyl-1,3,2-dioxaborolan-2-yl)-1,2,5,6-tetrahydropyridine carboxylate was prepared according to the procedures described by P. R. Eastwood, Tetrahedron Lett. 2000, 41, 19, 3705-3708 and references cited therein.

1-Bromo-2,4-difluoro-5-nitrobenzene: To a 0° C. mixture of 1-bromo-2,4-difluorobenzene (20.0 g; 11.7 mL; 0.100 mol) and H₂SO₄ (76.8 mL) was added HNO₃ (68.0 mL) over 45 min at such a rate that the internal temperature was <7° C. The resulting mixture was stirred for 1 h at 0° C., poured into ice water (400 mL), stirred vigorously for 2-3 min and extracted with CH₂Cl₂ (400 mL). The organic layers were washed with brine (1×500 mL), dried over Na₂SO₄, filtered and evaporated to give the product as a yellow oil (23.5 g, 95%). ¹H NMR (CDCl₃) δ 8.39 (t, J=7.2Hz, 1H), 7.14 (ddd, J=0.3, 7.8, 9.9Hz, 1H).

2-Bromo-5-fluoro-4-nitro toluene: To a mixture of nitronium tetrafluoroborate (11.6 g; 87.0 mmol) and CH₂Cl₂ (60.0 mL) was added 2-bromo-5-fluoro toluene (15.0 g, 10.0 mL, 79.0 mmol) over 5 min. After refluxing for 4.5 h, the mixture was cooled to room temperature and poured into ice water (150 mL). The mixture was extracted with CH₂Cl₂ (3 X 50 mL). The combined organic layers were washed with brine (100 mL), dried over Na₂SO₄, filtered and concentrated to give 18.3 g of crude product. The crude product was treated with hexane and cooled to −70° C. then the hexane was decanted away from the resulting solid to give 9.77 g of the desired product as a semi-solid (53%). The mother liquors were evaporated and purified by column chromatography (silica gel, 2% EtOAc in Hexane) to give 1.0 g of the desired product. ¹H NMR (CDCl₃) 6 8.26 (d, J=6.9Hz, 1H), 7.20 (d, J=11.7Hz, 1H), 2.48 (s, 3H).

1-Bromo-3-nitro-2,4,6-trifluorobenzene: To a cooled (1.3° C.) mixture of 1-bromo-2,4,6-trifluorobenzene (30.0 g; 142 mmol) and H₂SO₄ (115 mL) was added HNO₃ (68%; 102 mL) over 1.5 h at such a rate that the internal temperature was <8° C. After stirring at 0° C. for 2 h, the resulting mixture was poured into ice water (1850 mL), stirred vigorously for 30 min and extracted with CH₂Cl₂ (3×600 mL). The combined organic layers were washed with water (2×600 mL), dried over MgSO₄, filtered and concentrated to give the desired product as a clear yellow oil (35.0 g, 99%). ¹H NMR (CDCl₃) 6 7.01 (ddd, J=2.4, 7.8, 9.3Hz, 1H); ¹⁹F NMR (CDCl₃) δ−116.20 to −116.10, −107.73 to −107.71, −93.80 to −93.70.

3-Bromo-2-methyl-5-nitropyridine:

Step 1: A mixture of 2-hydroxy-5-nitropyridine (50.0 g; 0.358 mol) and water (7 L) was warmed to 40° C. and bromine (21.1 mL; 0.393 mol) was added dropwise over −20 min. After stirring at 40° C. for 2.5 h, the mixture was cooled to 10° C. and the crude product was isolated by filtration. The solid was washed with water and dried in vacuo to give 70.0 g of 3-bromo-2-hydroxy-5-nitropyridine as a solid (90% yield). mp 212-214 ° C. (with decomp); ¹H NMR (CD₃OD) 67 8.66 (d, J=2.9Hz, 1H), 8.64 (d, J=2.9Hz, 1H).

Step 2: To a cooled (0-5° C.) mixture of 3-bromo-2-hydroxy-5-nitropyridine (47.0 g; 0.214 mol) and quinoline (13.7 g; 0.107 mol) was added POCl₃ (26.0 mL; 0.278 mol) dropwise over ˜10 min (the mixture was difficult to stir initially but became less viscous as the reaction progressed and the mixture warmed). After stirring at 120° C. for 3.5 h, the mixture was cooled to 100° C. and water (90 mL) was added. The resulting mixture was stirred vigorously while cooling to 0-5° C. The product was collected by filtration, washed with water and dried in vacuo at 45° C. to give 42.0 g of 3-bromo-2-chloro-5-nitropyridine (82%). ¹H NMR (CD₃0D) δ 9.19 (d, J=2.4Hz, 1H), 8.93 (d, J=2.4Hz, 1H).

Step 3: To a cooled (15° C.) solution of diethyl malonate (8.8 mL; 58.0 mmol) in diethyl ether (110 mL) was added NaH (as a 60% dispersion in oil; 2.32 g; 58.0 mmol) over 5 min and 3-bromo-2-chloro-5-nitropyridine (12.5 g; 52.6 mmol) in four portions over ˜15 min (an exotherm to 26° C. was observed), followed by removal of diethyl ether in vacuo to give a red oil. After stirring the resulting red oil at 114° C. for 1 h 15 min, H₂SO₄ (6M, 67.0 mL) was added. The resulting mixture was heated at reflux for 8 h then cooled to 0° C. and the pH value was adjusted to 7 with 25% KOH aqueous solution (135 mL). The resulting mixture was stirred in an ice bath for 25 min and the crude product was collected and washed with water (50 mL) by filtration. The crude product was stirred in CH₂Cl₂ (350 mL) for 30 min and the impurity was removed by filtration. The organic layer was dried over Na₂SO₄, filtered and concentrated to give 11.1 g of the impure product as red oil. The red oil was dissolved in CH₂Cl₂ (100 mL) and hexanes (200 mL). The resulting mixture was filtered and the organic portion was concentrated to give 9.30 g of 3-bromo-2-methyl-5-nitropyridine as an orange crystalline solid (81%). ¹H NMR (CDCl₃) δ 9.25 (d, J=2.3Hz, 1H), 8.61 (d, J=2.3Hz, 1H), 2.80 (s, 3H).

Benzyl 5-bromo-3-pyridinyl carbamate: To a suspension of 5-bromonicotinic acid (20.0 g, 99.0 mmol) in toluene (200 mL) was added diphenylphosphoryl azide (25.6 mL, 118.8 mmol) and Et₃N (16.6 mL, 118.8 mmol). After stirring at room temperature for 30 min, benzyl alcohol (15.4 mL, 148.5 mmol) was added. The mixture was stirred at room temperature for 1 h then refluxed overnight. After cooling to room temperature, the reaction mixture was washed with H₂O, NaHCO₃ and brine, dried over MgSO₄ and concentrated. Purification by flash chromatography (15-50% EtOAc in Hexane) provided 22.2 g (72.5 mmol, 73%) of benzyl 5-bromo-3-pyridinylcarbamate: ¹H NMR (CDCl₃) 6 8.39-8.32 (m, 2H), 8.29 (s, 1H), 7.45-7.32 (m, 5H), 6.94 (s, 1H), 5.22 (s, 2H); ESMS m/e: 307.0 (M+H)⁺.

tert-Butyl 4-(3-aminophenyl)piperidine carboxylate, tert-butyl 4-(3-amino-4-fluorophenyl) piperidine carboxylate, tert-butyl 4-(3-amino-4,6-difluorophenyl)piperidine carboxylate were prepared according to the procedures described by M. R. Marzabadi et al. in PCT WO 2004/005257 A1 (pp. 48-82).

The following intermediates were prepared analogously:

tert-Butyl 4-(3-amino-6-methylphenyl)piperidinecarboxylate

¹H NMR (CDCl₃) δ 6.93 (d, J=8.1Hz, 1H), 6.53 (d, J=2.4Hz, 1H), 6.47 (dd, J=2.4, 8.1Hz, 1H), 4.30-4.18 (m, 2H),.3.53 (brs, 2H), 2.86-2.51 (m, 3H), 2.23 (s, 3H), 1.77-1.68 (m, 2H), 1.50-1.63 (m, 2H), 1.49 (s, 9H).

tert-Butyl 4-(3-amino-6-fluorophenyl)piperidinecarboxylate

¹H NMR (CDCl₃) δ 6.85-6.76 (m, 1H), 6.51-6.44 (m, 2H), 4.30-4.15 (m, 2H), 3.51 (brs, 2H), 2.98-2.73 (m, 3H), 1.82-1.73 (m, 2H), 1.66 -1.50 (m, 2H), 1.48 (s, 9H).

tert-Butyl 4-(3-amino-4-fluoro-6-methylphenyl)piperid inecarboxylate

¹H NMR (CDCl₃) δ 6.77 (d, J=12.0Hz, 1H), 6.60 (d, J=9.OHz, 1H), 4.32-4.16 (m, 2H), 3.86-3.52 (br, 2H), 2.86-2.67 (m, 3H), 2.22 (s, 3H), 1.69 (m, 2H), 1.60-1.43 (m, 11H).

tert-Butyl 4-(3-amino-2,4,6-trifluorophenyl)piperid inecarboxylate

¹H NMR (CDCl₃) δ 6.67-6.54 (m, 1H), 4.32-4.15 (m, 2H), 3.60-3.48 (m, 2H), 3.10-2.97 (m, 1H), 2.84-2.68 (m, 2H), 2.06-1.88 (m, 2H), 1.70-1.60 (m, 2H), 1.46 (s, 9H).

tert-Butyl 4-(5-amino-3-pyridyl) piperidinecarboxylate

¹H NMR (CDCl₃) δ 8.01-7.95 (m, 1H), 7.89 (s, 1H), 6.83 (s, 1H), 4.39-4.09 (br, 2H), 3.90-3.50 (br, 2H), 2.88-2.68 (m, 2H), 2.67-2.52 (m, 1H), 1.88-1.71 (m, 2H), 1.68-1.49 (m, 2H), 1.48 (s, 9H); ESMS m/e: 278.3 (M+H)⁺.

tert-Butyl 4-(5-amino-2-methyl-3-pyridyl)piperidinecarboxylate

¹H NMR (CDCl₃) δ 7.87 (d, J=2.7Hz, 1H), 6.80 (d, J=2.7Hz, 1H), 4.33-4.17 (m, 2H), 3.57-3.50 (br, 2H), 2.88-2.70 (m, 3H), 2.46 (s, 3H), 1.79-1.70 (m, 2H), 1.61-1.43 (m, 11H).

3-{1-[(4-arylthiophenyl)methyl]-4-pi peridyl}phenylamine synthesis:

3-(1-{[4-(4-methoxyphenylthio)phenyl]methyl}(4-piperdiyl))-4-methylphenylamine:

Step 1: Benzyl chloroformate (95%, 4.52 mL, 30.1 mmol) was added dropwise to a stirred mixture of tert-butyl 4-(3-amino-6-methylphenyl)piperidinecarboxylate (6.99 g, 24.1 mmol) and K₂CO₃ (3.66 g, 26.4 mmol) in tetrahydrofuran (350 mL) and stirred under nitrogen for 18 h. CH₂Cl₂ was added to the reaction mixture, washed with NaHCO₃ solution (saturated), followed by water, then dried over Na₂SO₂ and concentrated in vacuo to give an oil. Flash column chromatography (silica gel 60) eluting with cyclohexane:EtOAc (87:13) gave tert-butyl 4-{2-methyl-5-[(phenylmethoxy)carbonylamino]phenyl}piperidinecarboxylate (7.90 g, 77.1%) as a white foam. ESMS m/e: 425.0 (M+H)⁺.

Step 2: tert-Butyl 4-{2-methyl-5-[(phenylmethoxy)carbonylamino]phenyl}piperidine carboxylate (7.50 g, 17.6 mmol) was dissolved in CH₂Cl₂ (120 mL) and a HCl solution (4M in dioxane, 52.0 mL) was added and stirred for 1 h. The mixture was concentrated in vacuo and redissolved in CH₂Cl₂. Na₂CO₃ solution (1 M) was added and the biphasic mixture was stirred for 20 min and separated. The organic layer was further washed with water, dried over Na₂SO₄ and concentrated in vacuo to afford N-(4-methyl-3-(4-piperidyl)phenyl)(phenylmethoxy) carboxamide (5.61 g, 98%) as a white solid. ESMS m/e: 326.0 (M+H)⁺.

Step 3: A mixture of N-(4-methyl-3-(4-piperidyl)phenyl)(phenyl methoxy)carboxamide (324 mg, 1.00 mmol) and 4-(4-methoxyphenylthio)benzaldehyde (244 mg, 1.00 mmol), dichloroethane (5.00 mL), acetic acid (60.0 mg, 1.00 mmol) and sodium triacetoxborohydride (424 mg, 2.00 mmol was stirred under nitrogen at room temperature for 18 h. Saturated NaHCO₃ solution and CH₂Cl₂ was added. The mixture was separated, and the organic layer was washed with water (10 mL), dried over Na₂SO₄ and concentrated in vacuo. Flash column chromatography (silica gel 60) eluting with cyclohexane:EtOAc (85:15 then 7:3) gave N-[3-(1-{[4-(4-methoxyphenylthio)phenyl]methyl}(4-piperidyl))-4-methylphenyl](phenylmethoxy) carboxamide (486 mg, 88.0%) as a white foam. ESMS m/e:, 553.2 (M+H)⁺.

Step 4: N-[3-(1-{[4-(4-methoxyphenylthio)phenyl]methyl}(4-piperidyl))-4-methylphenyl] (phenylmethoxy)carboxamide (486 mg, 0.881 mmol) was dissolved in methanol (10.0 mL) and a KOH solution (40%, 1.20 mL) was added. The reaction mixture was heated at 100° C. for 10 h. After cooling to room temperature, the reaction mixture was poured into a separatory funnel. The phases were separated and the aqueous phase extracted with CH₂Cl₂ (2×20 mL) and the combined organic layers were washed with brine, dried over NaSO₄, filtered and concentrated in vacuo. Flash column chromatography (silica gel 60) eluting with Hexane:EtOAc (1:1 then 1:4) gave 3-(1-{[4-(4-methoxyphenylthio)phenyl]methyl}(4-piperidyl))-4-methylphenylamine (362 mg, 98.2%). ESMS m/e: 419.2 (M+H)⁺.

### EXAMPLES

### Example 1a

N-[3-(1-{[4-(4-fluorophenylthio)phenyl]methyl}(4-piperidyl))-4-methylphenyl]-2-methylpropanamide:

2-methyl-N-(4-methyl-3-(4-piperidyl)phenyl) propanamide (130 mg, 0.500 mmol) and 4-(4-fluorophenylthio)benzaldehyde (116 mg, 0.500 mmol) were dissolved in dichloroethane (5.00 mL) and acetic acid (30.0 mg, 0.500 mmol), and sodium triacetoxborohydride (212 mg, 1.00 mmol) was added at room temperature. Stirring was continued under nitrogen at room temperature for 10 h, then a saturated NaHCO₃ solution and CH₂Cl₂ was added. The mixture was separated, and the organic phase was washed with water, dried over Na₂SO₄ and concentrated in vacuo. Flash column chromatography (silica gel 60) eluting with cyclohexane:EtOAc (85:15 then 7:3) gave N-[3-(1-{[4-(4-fluorophenylthio)phenyl]methyl}(4-piperidyl))-4-methylphenyl]-2-methylpropanamide (86.3 mg, 36.2%) as a white solid. ESMS m/e: 477.2 (M+H)⁺.

The following intermediates were prepared analogously:

### Example 1b

N-[4-fluoro-3-(1-{[4-(4-fluorophenylthio)phenyl]methyl}(4-piperidyl))phenyl] (methylethoxy)carboxamide: ESMS m/e: 497.2 (M+H)⁺.

### Example 1c

cyclopropyl-N-[2-fluoro-5-(1-{[4-(4-fluorophenylthio)phenyl]methyl}(4-piperidyl))-4-methylphenyl]carboxamide: ESMS m/e: 493.3 (M+H)⁺.

### Example 1d

N-[3-(1-{[4-(4-chlorophenylthio)phenyl]methyl(4-piperdyl))-4-methylphenyl]-2-methylpropanamide: ESMS m/e: 493.2 (M+H)⁺.

### Example 1e

N-[5-(1-{[4-(4-chlorophenylthio)phenyl]methyl}(4-piperidyl))-2-fluoro-4-methyl phenyl]-2-methylpropanamide: ESMS m/e: 511.1 (M+H)⁺.

### Example 1f

N-[3-(1-{[4-(4-chlorophenylthio)phenyl]methyl}(4-piperidyl))-4-methylphenyl] methoxycarboxamide: ESMS m/e: 481.1 (M+H)⁺.

### Example 1g

N-[5-(1-{[4-(4-chlorophenylthio)phenyl]methyl}(4-piperidyl))-6-methyl(3-pyridyl)]cyclobutylcarboxamide: ESMS m/e: 506.2 (M+H)⁺.

### Example 1h

N-[5-(1-{[4-(4-chlorophenylthio)phenyl]methyl}(4-piperidyl))-6-methyl(3-pyridyl)]methoxycarboxamide: ESMS mle: 482.1 (M+H)⁺.

### Example 1i

(dimethylamino)-N-[3-(1-{[4-(4-chlorophenylthio)phenyl]methyl}(4-piperidyl))-4-methylphenyl]carboxamide: ESMS m/e: 494.2 (M+H)⁺.

### Example 1j

N-[3-(1-{[4-(4-chlorophenylthio)phenyl]methyl}(4-piperidyl))-2,4,6-trifluoro phenyl]-2-methylpropanamide: ESMS m/e: 533.2 (M+H)⁺.

### Example 1k

2-(dimethylamino)-N-[3-(1-{[4-(4-chlorophenylthio)phenyl]methyl}(4-piperidyl))-4-methylphenyl]acetamide: ESMS m/e: 508.2 (M+H)⁺.

### Example 1l

N-[3-(1-{[4-(3-fluorophenylthio)phenyl]methyl}(4-piperidyl))-4-methylphenyl]-2-methylpropanamide: ESMS m/e: 477.2 (M+H)⁺.

### Example 1m

N-[3-(1-{[4-(2,4-difluorophenylthio)phenyl]methyl}(4-piperidyl))-4-methyl phenyl]-2-methylpropanamide: ESMS m/e: 495.3 (M+H)⁺; Anal Calcd for C₂₉H₃₂F₂N₂0S+HCl+0.17CH₂Cl₂: C, 64.22; H, 6.16; N, 5.13. Found: C, 64.93; H, 6.15; N, 4.80.

### Example 1n

N-[3-(1-{[4-(3-chloro-4-fluorophenylthio)phenyl]methyl}(4-piperidyl))-4-methyl phenyl]-2-methylpropanamide: ESMS m/e: 511.2 (M+H)⁺.

### Example 1o

N-[3-(1-{[4-(2-fluorophenylthio)phenyl]methyl}(4-piperidyl))4-methylphenyl]-2-methylpropanamide: ESMS m/e: 477.2 (M+H)⁺.

### Example 1p

2-methyl-N-(4-methyl-3-{1-[(4-(2-pyridylthio)phenyl)methyl](4-piperidyl)} phenyl)propanamide: ESMS m/e: 460.3 (M+H)⁺; Anal Calcd for C₂₈H₃₃N₃0S+HCl+0.92CH₂Cl₂: C, 60.49; H, 6.29; N, 7.32. Found: C, 60.04; H, 6.29; N, 7.08.

### Example 1q

N-[3-(1-{[4-(4-methoxyphenylthio)phenyl]methyl}(4-piperidyl))4-methyl phenyl]-2-methylpropanamide: ESMS m/e: 489.2 (M+H)⁺; Anal Calcd for C₃₀H₃₆N₂0₂S+HCl+0.54CH₂Cl₂: C, 64.24; H, 6.72; N, 4.91. Found: C, 64.25; H, 6.71; N, 4.82.

### Example 1r

2-methyl-N-{4-methyl-3-[1-({4-[5-(trifluoromethyl)(2-pyridylthio)]phenyl} methyl)(4-piperidyl)]phenyl}propanamide: ESMS m/e: 528.2 (M+H)⁺.

### Example 1s

2-methyl-N-(4-methyl-3-{1-[(4-phenylthiophenyl)methyl](4-piperidyl)}phenyl)propanamide: ESMS m/e: 459.2 (M+H)⁺; Anal Calcd for C₂₉H34N₂0S+HCl+0.35CH₂Cl₂: C, 67.17; H, 6.86; N, 5.34. Found: C, 67.16; H, 6.93; N, 5.17.

### Example 1t

2-methyl-N-(4-methyl-3-1-[(4-(4-pyridylthio)phenyl)methyl](4-piperidyl)} phenyl)propanamide: ESMS m/e: 460.2 (M+H)⁺.

### Example 1u

N-{3-[1-({4-[4-(tert-butyi)phenylthio]phenyl}methyl)(4-piperidyl)]4-methyl phenyl}-2-methylpropanamide: ESMS m/e: 515.3 (M+H)⁺.

### Example 1v

N-{5-[1-({4-[(4-chlorophenyl)sulfinyl]phenyl}methyl)(4-piperidyl)]-6-methyl(3-pyridyl)}cyclobutylcarboxamide: ESMS m/e: 522.2 (M+H)⁺.

### Example 1w

N-{3-[1-({4-[(4-chlorophenyl)sulfinyl]phenyl}methyl)(4-piperidyl)]-4-methyl phenyl}-2-methylpropanamide: ESMS m/e: 509.2 (M+H)⁺.

### Example 1x

N-{5-[1-({4-[(4-chlorophenyl)sulfonyl]phenyl}methyl)(4-piperidyl)]-6-methyl(3-pyridyl)}cyclobutylcarboxamide: ESMS m/e: 538.2 (M+H)⁺.

### Example 1y

N-{5-[1-({4-[(4-chlorophenyl)sulfinyl]phenyl}(methyl)(4-piperidyl)]-6-methyl(3-pyridyl)}methoxycarboxamide: ESMS m/e: 498.2 (M+H)⁺.

### Example 1z

N-{5-[1-({4-[(4-chlorophenyl)sulfonyl]phenyl}methyl)(4-piperidyl)]-6-methyl(3-pyridyl)}methoxycarboxamide: ESMS m/e: 514.2 (M+H)⁺.

### Example 1aa

2-(dimethylamino)-N-{3-[1-({4-[(4-chlorophenyl)sulfinyl]phenyl}methyl)(4-piperidyl)]-4-methylphenyl}acetamide: ESMS m/e: 524.2 (M+H)⁺.

### Example 1bb

N-{3-[1-({4-[(4-chlorophenyl)sulfinyl]phenyl}methyl)(4-piperidyl)]-2,4,6-trifluorophenyl}-2-methylpropanamide: ESMS m/e: 549.1 (M+H)⁺.

### Example 2a

N-[3-(1-{[4-(4-methoxyphenylthio)phenyl]methyl}(4-piperidyl))-4-methyl phenyl]-2-(methylamino)acetamide:

To a stirred solution of 3-(1-{[4-(4-methoxyphenylthio)phenyl]methyl}(4-piperidyl))-4-methylphenylamine (121 mg, 0.289 mmol) in CH₂Cl₂/ dimethylformamide (2.00/0.200 mL) was added 2-[(tert-butoxy)-N-methylcarbonylamino]acetic acid (54.6 mg, 0.289 mmol) and 1-[3-(dimethylamino)propyl]-3-ethylcarbodiimide hydrochloride (111 mg, 0.578 mmol) and 4-dimethylaminopyridine (5.00 mg). The reaction mixture was stirred at room temperature for 10 h, then partitioned between CH₂Cl₂ (10 mL) and saturated NaHCO₃ solution (10 mL) and the organic phase was separated and washed with water (10 mL), then brine (10 mL), dried over MgSO₄ and concentrated in vacuo to give the crude product. Purification on silica gel (silica gel 60, 40 mL) eluting with CH₂Cl₂ then 3% methanol in CH₂Cl₂ gave 2-[(tert-butoxy)-N-methylcarbonylamino]-N-[3-(1-{[4-(4-methoxyphenylthio)phenyl]methyl}(4-piperidyl))-4-methylphenyl]acetamide (84.7 mg, 50.1%) as a yellow solid.

2-[(tert-butoxy)-N-methylcarbonylamino]-N-[3-(1-{[4-(4-methoxyphenylthio)phenyl]methyl}(4-piperidyl))-4-methylphenyl]acetamide was dissolved in CH₂Cl₂ (1.00 mL) and trifluoroacetic acid (0.160 mL) was added to the stirred solution. Stirring was continued for 10 min then the reaction mixture was concentrated in vacuo to give a gum. The crude product was dissolved in saturated NaHCO₃ solution (10 mL to pH 10) and extracted with CH₂Cl₂ (2×10 mL). The combined organic layers were dried over MgSO₄ and concentrated in vacuo to give N-[3-(1-{[4-(4-methoxyphenylthio)phenyl]methyl}(4-piperdyl))-4-methylphenyl]-2-(methylamino)acetamide (68.6 mg, 99.2%) as a yellow solid. ESMS m/e: 490.2 (M+H)⁺;

The following intermediates were prepared analogously:

### Example 2b

((2S)(2-piperidyl))-N-[3-(1-{[4-(4-methoxyphenylthio)phenyl]methyl}(4-piperidyl))-4-methylphenyl]carboxamide: ESMS m/e: 530.3 (M+H)⁺.

### Example 2c

N-[3-(1-{[4-(4-methoxyphenylthio)phenyl]methyl}(4-piperidyl))-4-methyl phenyl]-4-piperidylcarboxamide: ESMS m/e: 530.3 (M+H)⁺.

### Example 2d

N-[3-(1-{[4-(3,4-difluorophenylthio)phenyl]methyl}(4-piperidyl))-4-methyl phenyl]-2-(methylamino)acetamide: ESMS m/e: 496.2 (M+H)⁺.

### Example 2e

((2S)(2-piperidyl))-N-[3-(1-{[4-(3,4-difluorophenylthio)phenyl]methyl}(4-piperidyl))-4-methylphenyl]carboxamide: ESMS m/e: 536.3 (M+H)⁺.

### Example 2f

N-[3-(1-{[4-(3,4-difluorophenylthio)phenyl]methyl}(4-piperidyl))-4-methyl phenyl]-4-piperidylcarboxamide: ESMS m/e: 536.2 (M+H)⁺.

### Example 2g

2-(methylamino)-N-(4-methyl-3-{1-[(4-(2-pyridylthio)phenyl)methyl](4-piperidyl)}phenyl)acetamide: ESMS m/e: 461.2 (M+H)⁺.

### Example 2h

N-(4-methyl-3-{1-[(4-(2-pyridylthio)phenyl)methyl](4-piperidyl)}phenyl)-4-piperidylcarboxamide: ESMS m/e: 501.2 (M+H)⁺.

### Example 2i

((2S)(2-piperidyl))-N-(4-methyl-3-{1-[(4-(2-pyridylthio)phenyl)methyl](4-piperidyl)}phenyl)carboxamide: ESMS m/e: 501.2 (M+H)⁺.

### Example 2j

N-[3-(1-{[4-(4-chlorophenylthio)phenyl]methyl}(4-piperidyl))-4-methylphenyl]-2-(ethylamino)acetamide: ESMS m/e: 508.2 (M+H)⁺.

### Example 2k

((2R)(2-piperidyl))-N-[3-(1-{[4-(4-chlorophenylthio)phenyl]methyl}(4-piperidyl))-4-methylphenyl]carboxamide: ESMS m/e: 534.2 (M+H)⁺.

### Example 2l

N-[3-(1-{[4-(4-chlorophenylthio)phenyl]methyl}(4-piperdyl))-4-methylphenyl]-2-(methylamino)acetamide: ESMS m/e: 494.2 (M+H)⁺.

### Example 2m

N-[3-(1-{[4-(4-chlorophenylthio)phenyl]methyl}(4-piperidyl))-4-methylphenyl]-4-piperidylcarboxamide: ESMS m/e: 534.2 (M+H)⁺.

### Example 2n

N-{3-[1-({4-[(4-chlorophenyl)sulfinyl]phenyl}methyl)(4-piperidyl)]-4-methyl phenyl}-2-(methylamino)acetamide: ESMS m/e: 510.3 (M+H)⁺.

### Example 2o

N-{3-[1-({4-[(4-chlorophenyl)sulfonyl]phenyl}methyl)(4-piperidyl)]-4-methylphenyl}-2-(ethylamino)acetamide: ESMS m/e: 540.2 (M+H)⁺.

### Example 2p

N-{3-[1-({4-[(4-chlorophenyl)sulfonyl]phenyl}methyl)(4-piperidyl)]-4-methyl phenyl}-2-(methylamino)acetamide: ESMS m/e: 526.1 (M+H)⁺.

### Example 2q

N-{3-[1-({4-[(4-chlorophenyl)sulfonyl]phenyl}methyl)(4-piperidyl)]-4-methylphenyl}-4-piperidylcarboxamide: ESMS m/e: 566.2 (M+H)⁺.

### Example 3a

Cyclopropyl-N-{4-methyl-3-[1-({4-[(4-methylphenyl)sulfinyl] phenyl}methyl)(4-piperidyl)]phenyl}carboxamide:

Into a vial was added 4-(bromomethyl)-1-[(4-methylphenyl)sulfinyl]benzene (100 mg, 0.320 mmol), cyclopropyl-N-(4-methyl-3-(4-piperidyl)phenyl)carboxamide (50.0 mg, 0.190 mmol), potassium carbonate (60.0 mg, 0.430 mmol), Nal (30.0 mg, 0.200 mmol) and 2.50 mL of dimethylformamide. The mixture was stirred for 2-3 min at 25° C. and heated to 90° C. (oil bath). After stirring 12 h at 90° C., the mixture was allowed to cool to 25° C. and was diluted with 50 mL of EtOAc. The reaction solution was then washed with water (3×30 mL) and the aqueous solution was extracted with 30 mL of EtOAc. The organic solutions were combined and dried over MgSO₄. Removal of solvent in vacuo gave crude product that was purified by chromatography to afford cyclopropyl-N-{4-methyl-3-[1-({4-[(4-methylphenyl)sulfinyl]phenyl}methyl)(4-piperidyl)]phenyl}carboxamide with silica gel, 97% EtOAc:3% methanol (2 M ammonia) 36.0 mg, 39.1%. ESMS m/e: 487.2 (M+H)⁺. The following compounds were prepared analogously:

### Example 3b

2-methyl-N-[4-methyl-3-(1-{[4-(phenylsulfonyl)phenyl]methyl}(4-piperidyl)) phenyl]propanamide: ESMS m/e: 491.2 (M+H)⁺.

### Example 3c

N-[4-chloro-3-(1-{[4-(phenylsulfonyl)phenyl]methyl}(4-piperidyl))phenyl]-2-methylpropanamide: ESMS m/e: 511.2 (M+H)⁺.

### Example 3d

N-{5-[1-({4-[(4-chlorophenyl)sulfonyl]phenyl}methyl)(4-piperidyl)]-2-fluoro-4-methylphenyl}-2-methylpropanamide: ESMS m/e: 543.2 (M+H)⁺.

### Example 3e

cyclopropyl-N-[2-fluoro-4-methyl-5-(1-{[4-(phenylsulfonyl)phenyl]methyl}(4piperidyl))phenyl]carboxamide: ESMS m/e: 507.2 (M+H)⁺.

### Example 3f

cyclopropyl-N-{3-[1-({4-[(4-fluorophenyl)sulfonyl]phenyl}methyl)(4-piperidyl)]-4-methylphenyl}carboxamide: ESMS m/e: 507.3 (M+H)⁺.

### Example 3g

N-{3-[1-({4-[(4-chlorophenyl)sulfonyl]phenyl}methyl)(4-piperidyl)]-4-methyl phenyl}-2-methylpropanamide: ESMS m/e: 525.2 (M+H)⁺.

Formulations

The pharmaceutical formulations of the invention may be prepared by conventional methods in the art.

For example tablets may be prepared by mixing the active ingredient with ordinary adjuvants and/or diluents and subsequently compressing the mixture in a conventional tabletting machine prepare tablets. Examples of adjuvants or diluents comprise: corn starch, potato starch, talcum, magnesium stearate, gelatine, lactose, gums, and the like. Any other adjuvants or additives usually used for such purposes such as colorings, flavorings, preservatives etc. may be used provided that they are compatible with the active ingredients.

1) Tablets containing 5.0 mg of Compound 1k calculated as the free base:

| | | |
|---|---|---|
| Compound 1k | 5.0 | mg |
| Lactose | 60 | mg |
| Maize starch | 30 | mg |
| Hydroxypropylcellulose | 2.4 | mg |
| Microcrystalline cellulose | 19.2 | mg |
| Croscarmellose Sodium Type A | 2.4 | mg |
| Magnesium stearate | 0.84 | mg |

2) Tablets containing 0.5 mg of Compound 1k calculated as the free base:

| | | |
|---|---|---|
| Compound 1k | 0.5 | mg |
| Lactose | 46.9 | mg |
| Maize starch | 23.5 | mg |
| Povidone | 1.8 | mg |
| Microcrystalline cellulose | 14.4 | mg |
| Croscarmellose Sodium Type A | 1.8 | mg |
| Magnesium stearate | 0.63 | mg |

3) Syrup containing 25 mg of Compound 1k per milliliter:

| | | |
|---|---|---|
| Compound 1k | 25 | mg |
| Sorbitol | 500 | mg |
| Hydroxypropylcellulose | 15 | mg |
| Glycerol | 50 | mg |
| Methyl-paraben | 1 | mg |
| Propyl-paraben | 0.1 | mg |
| Ethanol | 0.005 | mL |
| Flavor | 0.05 | mg |
| Saccharin | 0.5 | mg |
| Water | 1 | mL |

In Vitro Methods

The pharmacological properties of the compounds of the present invention were evaluated at the cloned rat MCH1 receptor using the protocols disclosed in U.S. Pat. No. 6,727,264, the contents of which are hereby incorporated by reference.

Using this protocol, the inhibition by the compound of the binding to a radiolabeled ligand (tritiated SNAP-7941) to membranes of rat cloned MCH1 receptors expressed in CHO cells was determined in vitro. The radiochemical synthesis of tritiated SNAP-7941 was performed by Amersham Pharmacia Biotech, Cardiff, Wales.

Briefly, the affinity of the compounds was measured by their ability to displace tritiated SNAP-7941 by incubating rat MCH1 expressing membranes with the compound and radioligand at 25° C. for 90 min. Incubation was terminated by rapid vacuum filtration over GF/C glass fiber filters, presoaked in 5% PEI using 50 nM Tris pH 7.4 as wash buffer. In all experiments, nonspecific binding is defined using 10 pM of tritiated SNAP-7941.

The binding affinities for the compounds in the present invention, exemplified above, at the MCH1 receptor were determined to be 200 nM or less. For the majority of the compounds, the Ki values are 100 nM or less, and for a large group of compounds the Ki values are 10 nM or less.

What is claimed:

1. A compound having the structure: 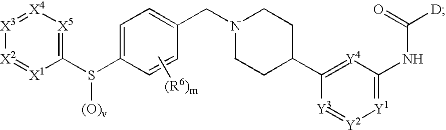
wherein each X¹, X², X³, X⁴ and X⁵ is independently CR¹ or N, provided that if one X is N then the remaining X are each CR¹;
wherein each y¹, y², y³ and Y⁴ is independently CR⁷ or N, provided that if one Y is N then the remaining Y are each CR⁷;
wherein D is 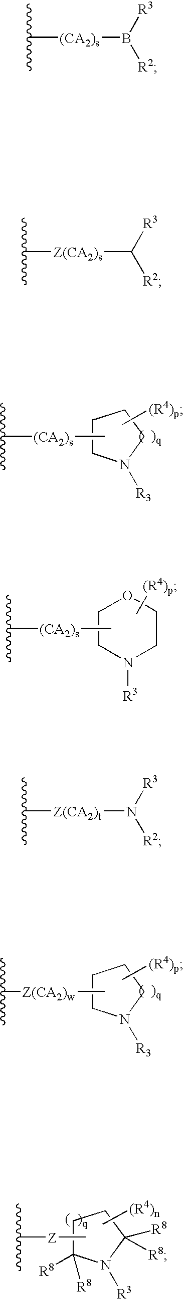 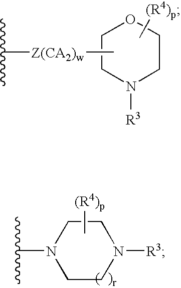
wherein Z is —N(R⁵) or —O—;
wherein each A is independently H or straight chained or branched C₁-C₄ alkyl;
wherein B is CH or N;
wherein each R¹ is independently H, straight chained or branched C₁-C₇ alkyl, straight chained or branched C₁-C₇ fluoroalkyl, straight chained or branched C₁-C₇ alkoxy, F, Cl, Br or I;
wherein R² is H or straight chained or branched C₁-C₄ alkyl;
wherein R³is H or straight chained or branched C₁-C₄ alkyl;
or wherein if B is N, then the R² moiety, B, the R³ moiety and a bond formed between the R² moiety and the R³ moiety form: 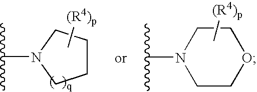
or wherein if B is CH, then the R² moiety, B, the R³ moiety and a bond formed between the R² moiety and the R³ moiety form cyclopropyl, cyclobutyl, cyclopentyl or cyclohexyl;
wherein R⁴ is H, straight chained or branched C₁-C₄ alkyl, straight chained or branched C₁-C₄ fluoroalkyl or F;
wherein R⁵ is H or straight chained or branched C₁-C₄ alkyl;
wherein each R⁶ is independently straight chained or branched C₁-C₇ alkyl, straight chained or branched C₁-C₇ fluoroalkyl, straight chained or branched C₁-C₇ alkoxy, F, Cl, Br or I;
wherein each R⁷ is independently H, straight chained or branched C₁-C₇ alkyl, straight chained or branched C₁-C₇ fluoroalkyl, straight chained or branched C₁-C₇ alkoxy, F, Cl, Br or I;
wherein R⁸ is H, straight chained or branched C₁-C₄ alkyl, straight chained or branched C₁-C₄ fluoroalkyl or F;
wherein m is an integer from 0 to 4 inclusive;
wherein n is an integer from 0 to 2 inclusive;
wherein p is an integer from 0 to 4 inclusive;
wherein q is an integer from 0 to 3 inclusive;
wherein r is 1 or 2;
wherein s is an integer from 0 to 4 inclusive;
wherein t is an integer from 2 to 4 inclusive;
wherein v is an integer from 0 to 2 inclusive; and
wherein w is an integer from 1 to 5 inclusive;
or a pharmaceutically acceptable salt thereof.

2. The compound of claim 1, wherein m is 0 or 1 and R⁶ is methyl, F or Cl.

3. The compound of claim 2, wherein each X¹, X², X³, X⁴ and X⁵ is CR¹, and each Y¹, Y², Y³ and Y⁴ is CR⁷.

4. The compound of claim 3, wherein each R¹ is independently H, methyl, F or Cl, and each R⁷ is independently H, F or methyl.

5. The compound of claim 4, wherein D is

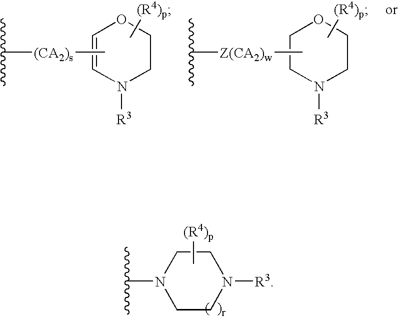

6. The compound of claim 5, wherein p is 0 and R₃ is H or methyl.

7. The compound of claim 4, wherein D is

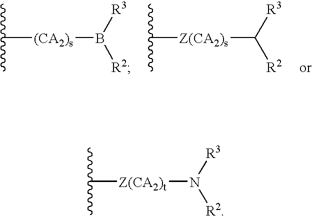

8. The compound of claim 7, wherein D is

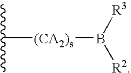

9. The compound of claim 8, wherein if B is N, then the R² moiety, B, the R³ moiety and the bond formed between the R² moiety and the R³ moiety form: 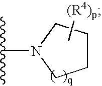
or wherein B is CH then the R² moiety, B, the R³ moiety and the bond formed between the R² moiety and the R³ moiety form cyclopropyl, cyclobutyl, cyclopentyl or cyclohexyl;

10. The compound of claim 8, wherein B is N, and R² and R³ are independently H, methyl or ethyl.

11. The compound of claim 10, wherein s is 1 or 2.

12. The compound of claim 7, wherein D is

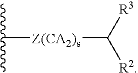

13. The compound of claim 12, wherein R² and R³ are independently H, methyl or ethyl.

14. The compound of claim 7, wherein D is

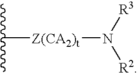

15. The compound of claim 14, wherein the R² moiety, N, the R³ moiety and the bond formed between the R² moiety and the R³ moiety form:

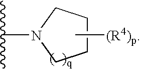

16. The compound of claim 14, wherein R² and R³ are independently H, methyl or ethyl.

17. The compound of claim 4, wherein D is 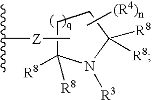
wherein n is 0 and R³ is H or methyl.

18. The compound of claim 4, wherein D is 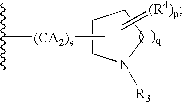
p is 0 and R³ is H or methyl.

19. The compound of claim 18, wherein s is 0 or 1.

20. The compound of claim 4, wherein D is 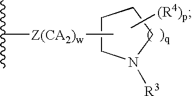
p is 0 and R³ is H or methyl.

21. A pharmaceutical composition comprising a therapeutically amount of a compound of claim 1 and a pharmaceutically acceptable carrier.

22. A process for making a pharmaceutical composition comprising mixing a therapeutically amount of a compound of claim 1 and a pharmaceutically acceptable carrier.

23. A method of treating a subject suffering from depression comprising administering to the subject a therapeutically effective amount of the compound of claim 1.

24. A method of treating a subject suffering from anxiety comprising administering to the subject a therapeutically effective amount of the compound of claim 1.
